(19) United States
(12) Patent Application Publication
(10) Pub. No.: US 2025/0001191 A1
(43) Pub. Date: Jan. 2, 2025

(54) POWER SOURCE AND IMPLANTABLE MEDICAL DEVICE INCLUDING SAME

(71) Applicant: Medtronic, Inc., Minneapolis, MN (US)

(72) Inventor: Christian S. Nielsen, River Falls, WI (US)

(21) Appl. No.: 18/754,763

(22) Filed: Jun. 26, 2024

Related U.S. Application Data
(60) Provisional application No. 63/523,950, filed on Jun. 29, 2023.

(51) Int. Cl. A61N 1/378; A61N 1/375

(52) U.S. Cl. CPC A61N 1/3785; A61N 1/37512; A61N 1/3754; A61N 1/3756

(57) ABSTRACT

Various embodiments of a power source and an implantable medical device that includes such power source are disclosed. The power source extends along a longitudinal axis between a first end and a second end of the power source. The power source further includes an outer housing extending along the longitudinal axis; an inner housing disposed at least partially within the outer housing and electrically isolated from the outer housing, where the inner housing extends along the longitudinal axis; and a passageway defined by an inner surface of the inner housing. The passageway extends between the first end and the second end of the power source.

### RELATED APPLICATIONS

This application claims the benefit of U.S. Provisional Application Ser. No. 63/523,950, filed Jun. 29, 2023, the entire contents of each of which are incorporated herein by reference.

### TECHNICAL FIELD

This disclosure generally relates to power sources and devices that utilize such power sources.

### BACKGROUND

Power sources such as batteries or electrochemical cells are generally used to provide power to devices when wired connections to external power sources can be undesirable or inconvenient. For example, batteries can be used to provide power to implantable medical devices implanted in a patient. In general, it can be desirable that such implantable medical devices be relatively small. However, as the size of implantable medical devices becomes smaller, the volume available to house a battery and electrical components also becomes smaller and more constricted. Accordingly, the geometry and size of the battery can contribute substantially to the overall size of implantable medical devices and electrical component placement therein as such devices become smaller.

### SUMMARY

In general, the present disclosure provides various embodiments of a power source (e.g., an electrical power source) and a device that utilizes such power source. The power source can include an outer housing and an inner housing disposed at least partially within the outer housing. The inner housing is electrically isolated from the outer housing. Further, an inner surface of the inner housing defines a passageway that extends between a first end and a second end of the power source. The passageway can allow electrical components to be disposed in or move through the power source via the passageway. Furthermore, the passageway can allow for wires or other electrical connections to be routed through the power source without or in addition to a feedthrough connection.

This disclosure includes without limitation the following clauses:

Clause 1: An electrical power source extending along a longitudinal axis between a first end and a second end of the power source. The power source includes an outer housing extending along the longitudinal axis; an inner housing disposed at least partially within the outer housing and electrically isolated from the outer housing, where the inner housing extends along the longitudinal axis; and a passageway defined by an inner surface of the inner housing. The passageway extends between the first end and the second end of the power source.

Clause 2: The power source of Clause 1, further including a first outer flange connected to the outer housing at the first end of the power source and a second outer flange connected to the outer housing at the second end of the power source.

Clause 3: The power source of Clause 2, further including a first inner flange connected to the inner housing at the first end of the power source and a second inner flange connected to the inner housing at the second end of the power source.

Clause 4: The power source of Clause 3, further including a first insulator disposed between the first outer flange and the first inner flange, and a second insulator disposed between the second outer flange and the second inner flange.

Clause 5: The power source of Clause 4, where at least one of the first insulator or second insulator includes a glass feedthrough material.

Clause 6: The power source of Clause 4, where at least one of the first insulator or second insulator includes a diffusion bonded sapphire material.

Clause 7: The power source of Clause 4, where at least one of the first insulator or second insulator includes a polymer material.

Clause 8: The power source of any one of Clauses 1-7, further including a kinetic energy harvester disposed at least partially within the passageway.

Clause 9: The power source of any one of Clauses 1-8, further including a power management system.

Clause 10: The power source of any one of Clauses 1-9, further including at least one conductor disposed at least partially within the passageway.

Clause 11: The power source of any one of Clauses 1-10, further including an anode, a cathode, and an electrolyte, where the anode, cathode, and electrolyte are disposed between the outer housing and the inner housing.

Clause 12: An implantable medical device including a device housing extending along a device axis between a first end and a second end of the device, and an electrical power source extending along a longitudinal axis between a first end and a second end of the power source.

The power source includes an outer housing extending along the longitudinal axis, where the outer housing defines at least a portion of the device housing; an inner housing disposed at least partially within the outer housing and electrically isolated from the outer housing, where the inner housing extends along the longitudinal axis; and a passageway defined by an inner surface of the inner housing, where the passageway extends between the first end and the second end of the power source.

Clause 13: The device of Clause 12, where the passageway of the power source extends along the longitudinal axis.

Clause 14: The device of Clause 12, where the passageway of the power source is substantially parallel to the longitudinal axis.

Clause 15: The device of any one of Clauses 12-14, further including a kinetic energy harvester disposed at least partially within the passageway.

Clause 16: The device of any one of Clauses 12-15, where the device further includes a first outer flange connected to the outer housing at the first end of the power source and a second outer flange connected to the outer housing at the second end of the power source.

Clause 17: The device of Clause 16, where the power source further includes a first inner flange connected to the inner housing at the first end of the power source and a second inner flange connected to the inner housing at the second end of the power source.

Clause 18: The device of Clause 17, where the power source further includes a first insulator disposed between the first outer flange and the first inner flange, and a second insulator disposed between the second outer flange and the second inner flange.

Clause 19: The device of Clause 18, where at least one of the first insulator or second insulator includes a glass feedthrough material.

Clause 20: The device of Clause 18, where at least one of the first insulator or second insulator includes a diffusion bonded sapphire material.

Clause 21: The device of Clause 18, where each of the first insulator or second insulator includes a polymer material.

Clause 22: The device of any one of Clauses 12-21, further including a power management system electrically connected to the power source.

Clause 23: The device of any one of Clauses 12-22, further including at least one conductor disposed at least partially within the passageway.

Clause 24: The device of any one of Clauses 12-23, where the power source further includes an anode, a cathode, and an electrolyte, where the anode, cathode, and electrolyte are disposed between the outer housing and the inner housing.

Clause 25: The device of any one of Clauses 12-24, where the implantable medical device is a leadless pacing device.

Clause 26: The device of any one of Clauses 12-25, where the passageway includes a diameter of at least 0.5 mm and no greater than 5.0 mm.

Clause 27: The device of any one of Clauses 12-26, further including a hermetic seal between the device housing and the outer housing of the power source.

Clause 28: The device of any one of Clauses 12-27, further including an electronic component disposed within the device housing and electrically coupled to the power source.

Clause 29: A method of forming an electrical power source, where the power source extends along a longitudinal axis between a first end and a second end of the power source. The method includes disposing an inner housing at least partially within an outer housing; disposing an anode, a cathode, and an electrolyte between an inner surface of the outer housing and an outer surface of the inner housing, where an inner surface of the inner housing defines a passageway that extends between the first end and the second end of the power source; and electrically isolating the inner housing from the outer housing.

Clause 30: The method of Clause 29, further including connecting a first outer flange to the outer housing at the first end of the power source and a first inner flange to the inner housing at the first end of the power source.

Clause 31: The method of Clause 30, further including connecting a second outer flange to the outer housing at the second end of the power source and a second inner flange to the inner housing at the second end of the power source.

Clause 32: The method of Clause 31, further including disposing a first insulator between the first outer flange and the first inner flange such that the first outer flange and first inner flange are electrically isolated, and disposing a second insulator between the second outer flange and the second inner flange such that the second outer flange and the second inner flange are electrically isolated.

Clause 33: The method of Clause 32, where at least one of the first insulator or second insulator includes a glass feedthrough material.

Clause 34: The method of Clause 32, where at least one of the first insulator or second insulator includes a diffusion bonded sapphire material.

Clause 35: The method of Clause 32, where at least one of the first insulator or second insulator includes a polymer material.

Clause 36: The method of any one of Clauses 29-35, further including disposing a kinetic energy harvester at least partially within the passageway.

Clause 37: The method of any one of Clauses 29-36, further including disposing at least one conductor at least partially within the passageway.

Clause 38: The method of any one of Clauses 29-37, further including connecting the outer housing to a device housing of an implantable medical device.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic perspective view of one embodiment of a power source.

FIG. 2 is a schematic cross-section view of the power source of FIG. 1.

FIG. 3 is a schematic cross-section view of the power source of FIG. 1 with a component disposed at least partially within a passageway of the power source.

FIG. 4 is a conceptual view of the power source of FIG. 1 with outer and inner housings removed for descriptive purposes.

FIG. 5 is an exploded view of the power source of FIG. 1.

FIG. 6 is a conceptual view of one embodiment of an implantable medical device including the power source of FIG. 1 disposed in a patient.

FIG. 7 is a perspective view of the implantable medical device of FIG. 6.

FIG. 8 is a schematic perspective view of the implantable medical device of FIG. 6 with a device housing of the device made transparent for descriptive purposes.

FIG. 9 is a flowchart of one embodiment of a method of manufacturing the power source of FIG. 1.

### DETAILED DESCRIPTION

In general, the present disclosure provides various embodiments of a power source (e.g., an electrical power source) and a device that utilizes such power source. The power source can include an outer housing and an inner housing disposed at least partially within the outer housing. The inner housing is electrically isolated from the outer housing. Further, an inner surface of the inner housing defines a passageway that extends between a first end and a second end of the power source. The passageway can allow electrical components to be disposed in or move through the power source via the passageway. Furthermore, the passageway can allow for wires or other electrical connections to be routed through the power source without or in addition to a feedthrough connection.

As electronic devices become smaller, the volume available to house batteries and electrical components also becomes smaller and more constricted. To minimize device size, a portion of an exterior surface of a given device can be defined or formed by a power source (e.g., battery) of such device. However, when a power source of a device defines a portion of an exterior surface of the device, the arrangement of components within a housing of the device can become even more restricted. For example, if an outer diameter or perimeter of a device is defined by a power source, electrical components can be restricted to a single side of the housing of the power source, or one or more electrical feedthroughs can be needed to allow electrical connections between devices on opposite sides of the power source. Furthermore, movement of components designed to move within the housing of the device can be limited by the lack of any pathway to move around or through the power source within the housing.

For example, a device can include an energy accumulator or harvester (e.g., a kinetic energy harvester) that is configured to convert mechanical energy to electrical energy. Such accumulator can be a capacitor, a battery, or both. These accumulators, however, occupy limited space within a device, thereby requiring other components of the device such as a power source to be further miniaturized.

Various embodiments of a power source and a device including such power source described herein can exhibit various advantages over typical power sources and devices. For example, a power source that includes a passageway that extends through the power source can allow for more flexibility in the arrangement of components within the device when the power source defines a portion of an exterior surface of the device. Power sources that include a passageway as described herein can allow electrical connections to be routed through the passageway, can allow components to be disposed in the passageway, or can allow components to move within or through the passageway. Furthermore, such power sources can allow the construction of smaller devices that retain at least some of the flexibility of component arrangement afforded by the space around power sources of larger devices.

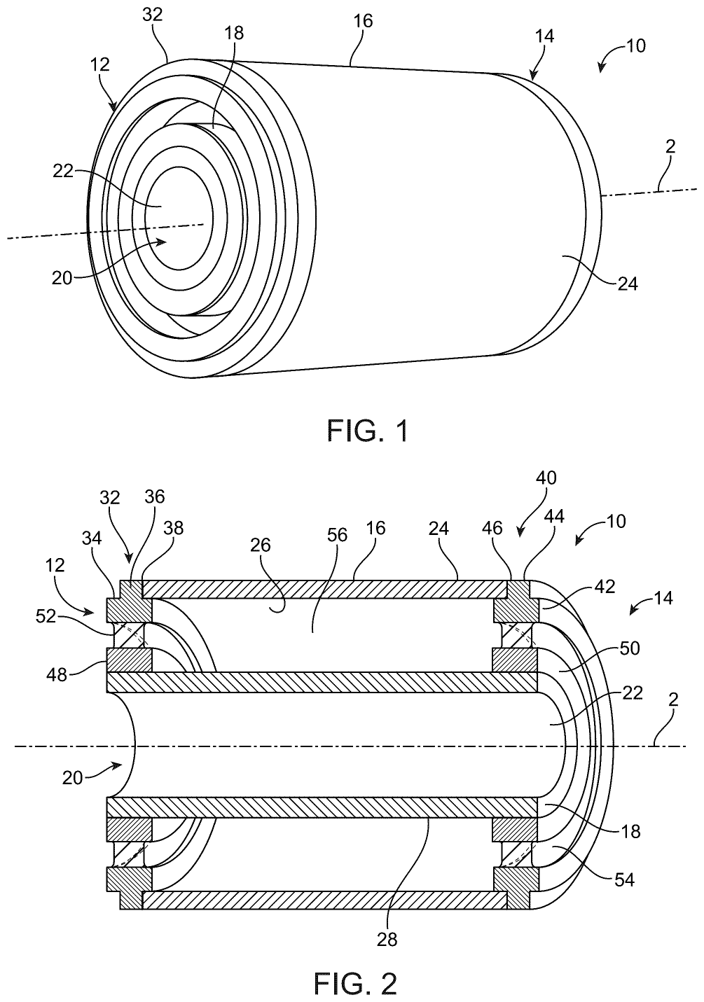

FIGS. 1-5 are various views of one embodiment of a power source 10. The power source 10 can include any suitable power source, e.g., an electrical power source. The power source 10 extends along a longitudinal axis 2 between a first end 12 and a second end 14 of the power source. The power source 10 includes an outer housing 16 extending along the longitudinal axis 2 and an inner housing 18 disposed at least partially within the outer housing and electrically isolated from the outer housing, where the inner housing extends along the longitudinal axis. A passageway 20 is defined by an inner surface 22 of the inner housing 18. The passageway 20 extends between the first end 12 and the second end 14 of the power source 10.

The power source 10 can be any suitable power source or sources, e.g., an electrical power source such as an electrochemical cell, a battery, etc. The power source 10 can be any suitable battery, e.g., a lithium-ion battery, a lithium metal battery, a lithium polymer battery, or other lithium batteries. Additionally, the power source 10 can be a primary cell (e.g., non-rechargeable) or a secondary cell (e.g., rechargeable). Further, the power source 10 can take any suitable shape and have any suitable dimensions.

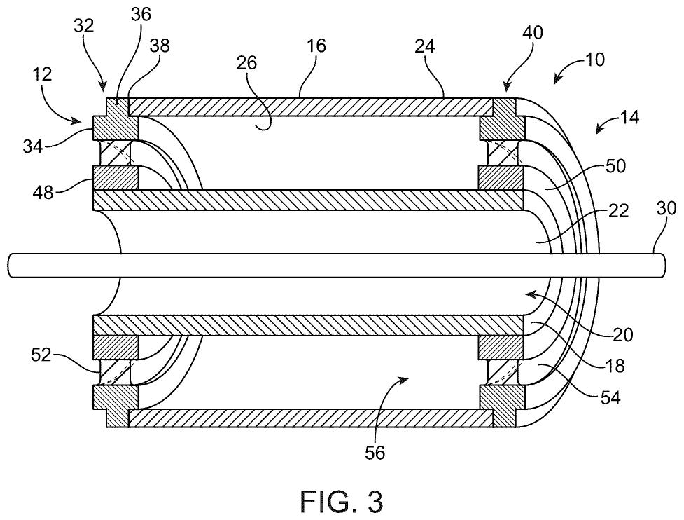

The outer housing 16 of the power source can take any suitable shape and have any suitable dimensions. The outer housing 16 can have a constant cross-sectional area along the longitudinal axis 2. In one or more embodiments, the outer housing 16 can have a cross-sectional area that varies along the axis 2. In one or more embodiments, an outer surface 24 of the outer housing 16 can define an exterior surface of the power source 10. Further, the outer housing 16 can define the general shape of the power source 10. For example, the outer housing 16 can define a cylinder, a polyhedron, a frustum, a pyramid, a cone, or other 3-dimensional shape. The outer housing 16 further includes an inner surface 26 (FIG. 3). The outer housing 16 can include suitable material, e.g., at least one of a metallic, polymer, ceramics such as alumina, polycrystalline or single crystal ruby, suitable glasses, polycrystalline or single crystal sapphire, etc. In one or more embodiments, the outer housing 16 can include a conductive material.

Disposed at least partially within the outer housing 16 is the inner housing 18. In one or more embodiments, the inner housing 18 is disposed entirely within the outer housing 16. The inner housing 18 can take any suitable shape and have any suitable dimensions. In one or more embodiments, the inner housing 18 can take the same shape as the outer housing 16. The inner housing 18 can have a constant cross-sectional area along the longitudinal axis 2. In one or more embodiments, the inner housing 18 can have a cross-sectional area that varies along the axis 2.

The inner housing 18 includes an outer surface 28 and the inner surface 22 (FIG. 2). The inner surface 22 defines the passageway 20 that extends between the first end 12 and the second end 14 of the power source 10. The inner housing 18 can include any suitable material, e.g., the same material described herein regarding the outer housing 16. In one or more embodiments, the outer housing 16 and the inner housing 18 include the same materials. In one or more embodiments, the outer housing 16 and the inner housing 18 include at least one different material. In one or more embodiments, the inner housing 18 includes a conductive material.

The inner surface 22 of the inner housing 18 defines the passageway 20 that extends between the first end 12 and the second end 14 of the power source 10. In one or more embodiments, the passageway 20 is substantially parallel to the longitudinal axis 2. In one or more embodiments, the passageway 20 extends along the longitudinal axis 2. The passageway 20 can take any suitable shape in a plane orthogonal to the longitudinal axis 2. In one or more embodiments, the passageway 20 has a constant cross-sectional area along the longitudinal axis 2. In one or more embodiments, the passageway 20 has a cross-sectional area the varies along the axis 2. In one or more embodiments, the passageway 20 can have a diameter or width of at least 0.5 mm and no greater than 5 mm.

As is further described herein, any suitable element or component can be disposed at least partially within the passageway 20. For example, as shown in FIG. 3, component 30 is disposed within the passageway 20. The component 30 can be disposed entirely within the passageway 20 or extend through the passageway. Any suitable number of components 30 can be disposed at least partially within the passageway 20. The component 30 can include any suitable element or device, e.g., at least one of a conductor, an electronic component (e.g., at least one of a capacitor, integrated circuit, processor, etc.), a kinetic energy harvester, etc. For example, the component 30 can be a moveable element of an energy harvester, such as one or more beams, which beam(s) can include a weight or proof mass at a distal (unsupported) end thereof. Such beam(s) can deflect or oscillate in bending/deflection in response to motion imparted to the device or energy harvester, such as cardiac motion. In one or more embodiments, the component 30 can be electrically connected to the inner housing 18. In one or more embodiments, the component 30 can be electrically isolated from the inner housing 18 using any suitable technique. The component 30 can include any suitable number of devices, circuitry, etc. For example, in one or more embodiments, the component 30 can include at least one conductor.

Connected to the outer housing 16 at the first end 12 of the power source 10 is a first outer flange 32. The first outer flange 32 can take any suitable shape and have any suitable dimensions. The first outer flange 32 can be a single part or multiple parts each connected to the outer housing 16 and/or to each other. In one or more embodiments, the first outer flange 32 can be integral with the outer housing 16, i.e., manufactured as a single part using any suitable technique, e.g., molding, machining 3D printing, etc. As shown, the first outer flange 32 includes a body 34 and a ledge 36 extending from the body. The first outer flange 32 is configured to connect the power source 10 to a housing of a device as is further described herein. Further, the ledge 36 of the first outer flange 32 is configured to mate with a first end 38 of the outer housing 16.

The first outer flange 32 can include any suitable material, e.g., the same material described herein regarding the outer housing 16. The first outer flange 32 can include the same material as the outer housing 16 or at least one material that is different from the material of the outer housing. In one or more embodiments, the first outer flange 32 can include a conductive material.

The first outer flange 32 can be connected to the outer housing 16 using any suitable technique, e.g., welding, bonding, laser welding, diffusion-assisted laser bonding, friction welding, etc. In one or more embodiments, the first outer flange 32 can be hermetically sealed to the outer housing 16 using any suitable technique, e.g., laser bonding.

The power source 10 also includes a second outer flange 40 connected to the outer housing 16 at the second end 14 of the power source. Although depicted as including first and second outer flanges 32, 40, the power source 10 can include only one outer flange or no outer flanges. The second outer flange 40 can take any suitable shape and have any suitable dimensions, e.g., the same shape and dimensions described herein regarding the first outer flange 32. The second outer flange 40 is configured to connect the power source 10 to a housing of a device or component as is further described herein. The second outer flange 40 can include a body 42 and a ledge 44 extending from the body (FIG. 2). The ledge 44 of the second outer flange 40 is configured to mate with a second end 46 of the outer housing 16.

The second outer flange 40 can include any suitable material, e.g., the same material described herein regarding the outer housing 16. The second outer flange 40 can include the same material as the outer housing 16 or at least one material that is different from the material of the outer housing. In one or more embodiments, the second outer flange 40 can include a conductive material.

The second outer flange 40 can be connected to the outer housing 16 using any suitable technique, e.g., the same techniques described herein regarding connection of the first outer flange 32 and the outer housing. Further, in one or more embodiments, the second outer flange 40 can be hermetically sealed to the outer housing 16 using any suitable technique, e.g., laser bonding.

The power source 10 can further include a first inner flange 48 connected to the inner housing 18 at the first end 12 of the power source and a second inner flange 50 connected to the inner housing at the second end 14 of the power source. Although depicted as including first and second inner flanges 48, 50, the power source 10 can include only one inner flange or no inner flanges. The first and second inner flanges 48, 50 can each take any suitable shape and have any suitable dimensions. Further, each of the flanges 48, 50 can be a single part or multiple parts each connected to the inner housing 18. In one or more embodiments, at least one of the first inner flange 48 or the second inner flange 50 can be integral with the inner housing 18, i.e., manufactured as a single part using any suitable technique, e.g., molding, machining 3D printing, etc.

The first and second inner flanges 48, 50 can each include any suitable material, e.g., the same material described herein regarding the outer housing 16. In one or more embodiments, the material of at least one of the first or second inner flanges 48, 50 is the same as the material of the inner housing 18.

Each of the first and second inner flanges 48, 50 can be connected to the inner housing 18 using any suitable technique, e.g., the same techniques described herein regarding connection of the outer flanges 32, 40 to the outer housing 16.

The inner housing 18 can be electrically isolated from the outer housing 16. As used herein, the term “electrically isolated” means that there is no current path from the outer housing to the inner housing or vice versa. Any suitable technique can be utilized to electrically isolate the outer housing 16 from the inner housing 18. For example, as shown in FIGS. 2-3, a first insulator 52 can be disposed between the first outer flange 32 and the first inner flange 48, and a second insulator 54 can be disposed between the second outer flange 40 and a second inner flange 50. In one or more embodiments, the first and second insulators 52, 54 can be utilized to connect the outer housing 16 to the inner housing 18 such that the outer and inner housings are spaced apart. The first and second insulators 52, 54 can include any suitable dielectric material that is configured to electrically isolate the outer housing 16 and the inner housing 18. In one or more embodiments, at least one of the first insulator 52 or second insulator 54 can include at least one of a glass feedthrough material, a diffusion bonded sapphire material, a polymer material, or a combination thereof. The first and second insulators 52, 54 can be connected to the outer flanges 32, 40 and inner flanges 48, 50 using any suitable technique. In one or more embodiments, the first and second insulators 52, 54 can hermetically seal a cavity 56 defined between the inner surface 26 of the outer housing 16 and the outer surface 28 of the inner housing 18. In one or more embodiments, the cavity 56 is hermetically sealed such that no passages or feedthroughs extend through the cavity. In such embodiments, any mechanical, optical, or electrical communication that extends through the power source 10 can be achieved via the passageway 20.

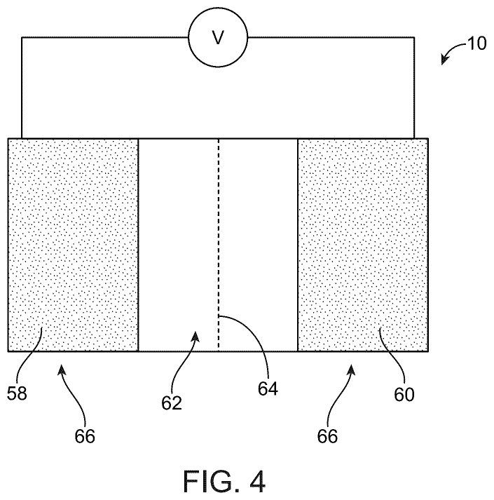

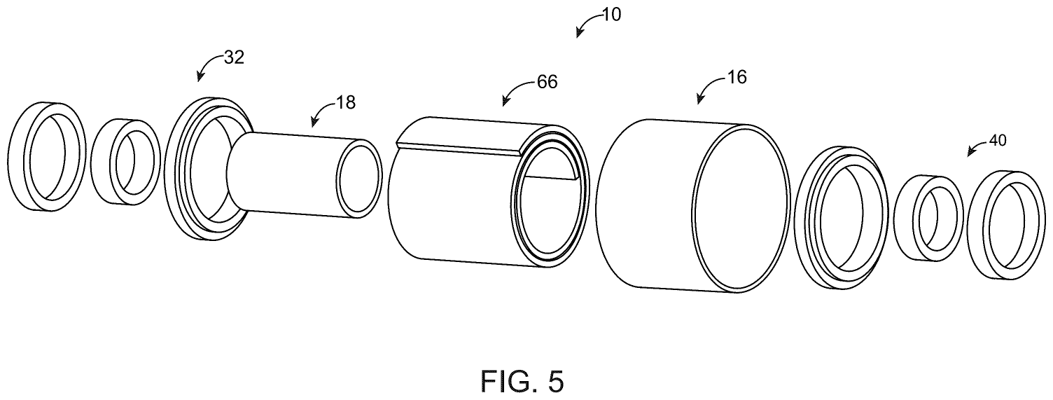

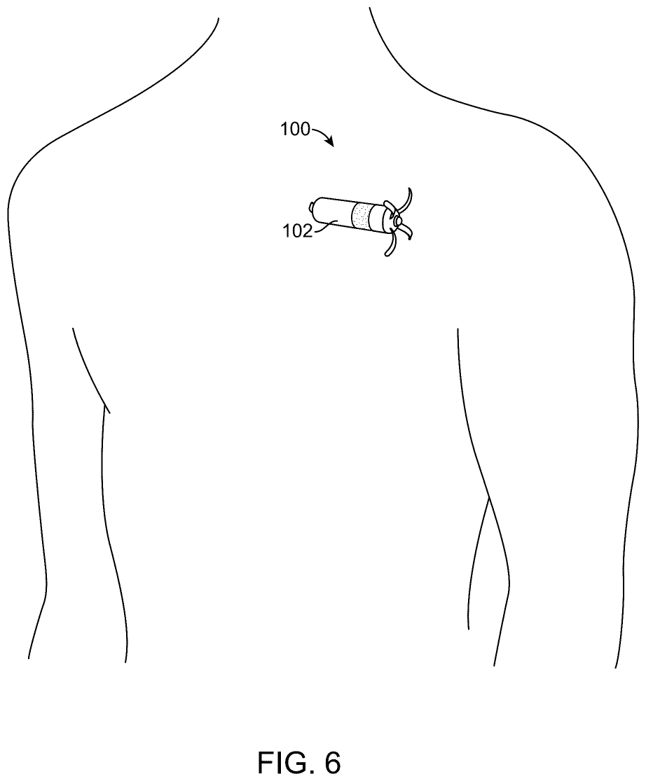

As mentioned herein, the power source 10 can include any suitable device capable of providing electrical energy to a device or system that is electrically connected to the power source. For example, FIG. 4 is schematic view of the power source 10 with the outer housing 16, inner housing 18, outer flanges 32, 40, and inner flanges 48, 50 removed for descriptive purposes. The power source 10 includes an anode 58, a cathode 60, an electrolyte 62, and a separator 64. In one or more embodiments, the anode 58, cathode 60, electrolyte 62, and separator 64 can be disposed in the cavity 56 defined by the inner surface 22 of the outer housing 16 and the outer surface 28 of the inner housing 18. The anode 58 and cathode 60 can be referred to collectively as electrodes 66. The electrodes 66 can be wrapped or wound in a spiral or other configuration (e.g., an oval or other hollow shape) around the outer surface 28 of the inner housing 18 as shown in FIG. 5, which is an exploded view of the power source 10. In a spiral configuration, the anode 58 or the cathode 60 can be wrapped around itself to form a plurality of layers or windings. However, the anode 58 or the cathode 60 can be wrapped to form a hollow shape that does not overlap itself and does not define a plurality of windings. Alternatively, one or both of the electrodes 66 can define a hollow shape that surrounds the inner housing 18. Accordingly, the electrodes 66 that define such a hollow shape can be disposed or provided around the inner housing 18 without being wrapped around such inner housing.

The electrodes 66 can include any suitable conductive or active materials. The particular conductive or active materials can depend on the type of the power source 10. In general, the anode 58 and the cathode 60 can be arranged such that they do not directly contact each other.

The power source 10 can also include a separator 64 arranged between the anode 58 and the cathode 60. In other words, the separator 64 can be provided intermediate to the anode 58 and the cathode 60. The separator 64 can be configured to prevent direct contact between the anode 58 and the cathode 60. The separator 64 can further be configured to allow transport of ionic charge carriers between the anode 58 and the cathode 60. The separator 64 can define a membrane that forms a microporous layer. The separator 64 can include any suitable material. The separator 64 can include, for example, one or more of a polymer, polyethylene, polypropylene, polyimide, cellulose, or other materials for forming a microporous layer.

The electrolyte 62 can transport positively charged ions between the anode 58 and the cathode 60. The electrolyte 62 can be disposed in the cavity 56 after the first and second insulators 52, 54 have been disposed between outer and inner flanges 32, 40, 48 50 using a fill port (not shown). The electrolyte 62 can include any suitable material such as, for example, lithium salts, lithium bis(trifluoromethylsulfonyl) imide (LiTFSI), lithium bis(pentafluoroethylsulfonyl) imide (LiBETI), lithium tris(trifluorosulfonyl) methide, lithium perchlorate (LiCIO4), lithium tetrafluoroborate (LiBF4), lithium hexafluoroarsenate (LiAsF6), lithium hexafluorophosphate (LiPF6), or other solute capable of transporting ionic charge carriers. The electrolyte 62 can be a liquid electrolyte.

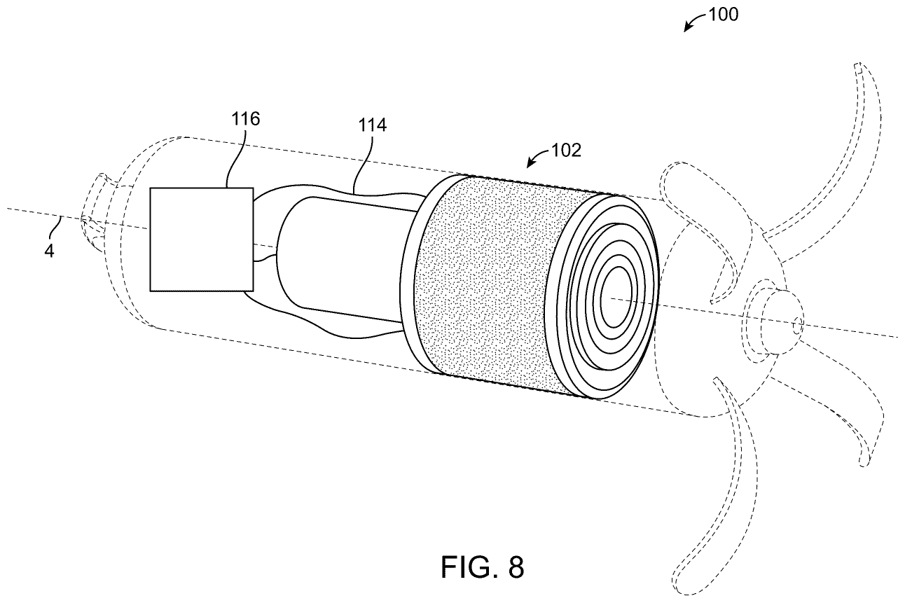

As mentioned herein, the power source 10 can be utilized to provide electrical energy to any suitable device or system. For example, FIGS. 6-8 are various views of one embodiment of an implantable medical device 100 that utilizes the power source 10 of FIGS. 1-5. The device 100 includes a device housing 102 that extends along a device axis 4 between a first end 104 and a second end 106 of the device. The device 100 can be any suitable electronic device or apparatus such as, for example, an implantable medical device, a sensor apparatus, a miniature camera, or other battery powered electronic device. In one or more embodiments, the device 100 can be a leadless pacing device.

The device axis 4 of the device 100 can be substantially parallel to the longitudinal axis 2 of the power source 10. In one or more embodiments, the device axis 4 and the longitudinal axis 2 can be substantially colinear.

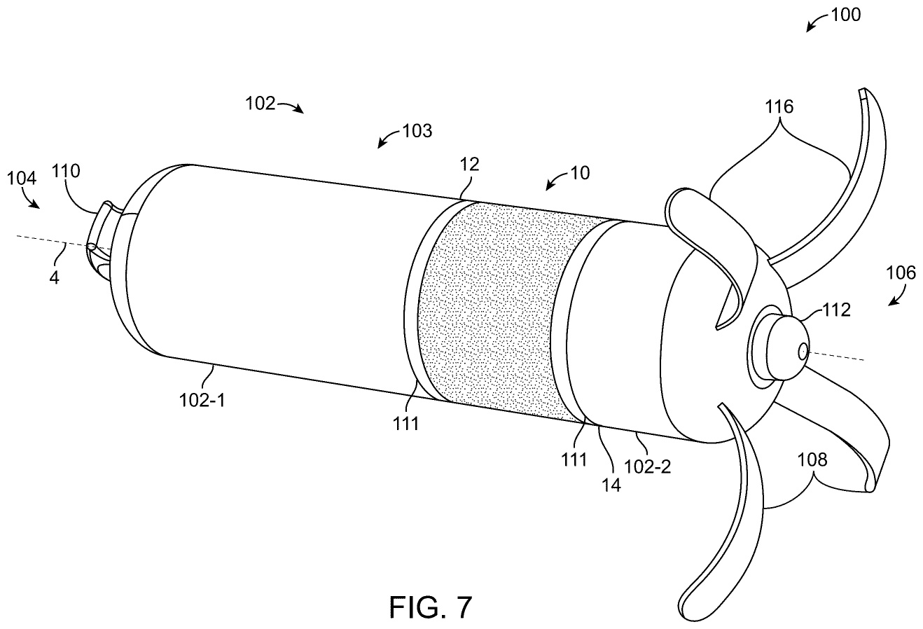

The device housing 102 and the outer housing 16 of the power source 10 define an exterior surface 103 of the device 100. The device housing 102 can include any suitable number of portions. As shown in FIG. 7, the device housing 102 includes a first portion 102-1 and a second portion 102-2 (collectively the device housing 102) that can be disposed in any suitable relationship. In the illustrated embodiment, the first portion 102-1 is connected to the first end 12 of the power source 10 and the second portion 102-2 is connected to the second end 14 of the power source. The portions of the device housing 102 can be connected to the power source 10 using any suitable technique, e.g., the same techniques described herein regarding connection of the outer flanges 32, 40 to the outer housing 16 of the power source. In general, the device housing 102 combined with the outer housing 16 of the power source 10 can define the overall or general shape of the device 100. Furthermore, the device housing 102 and the outer housing 16 can provide an interior space or volume for additional components of the device 100 and protection from the environment for these additional components. For example, the device housing 102 and the outer housing 16 can protect against dust or fluid ingress into the interior space of volume of the device 100. In one or more embodiments, the device 100 can include one or more hermetic seals 111 between the device housing 102 and the outer housing 16.

As shown, the housing 102 includes the first portion 102-1 and the second portion 102-2. The first portion 102-1 can extend along the device axis 4 from the outer housing 16 towards the first end 104 of the device 100. The second portion 102-2 can extend along the device axis 4 from the outer housing 16 towards the second end 106 of the device 100. In other words, the power source 10 can be arranged somewhere between the first end 104 and the second end 106 of the device 100 but does not define either of the first end 104 or the second end 106. In one or more embodiments, the power source 12 can define the first end 104 or the second end 106 of the device 100, and one or more portions of the device housing 102 can extend from the power source towards the other of the first end 104 and the second end 106. The hermetic seal 111 can be disposed between each of first portion 102-1 and second portion 102-2 of the device housing 102 and the outer housing 16. The hermetic seal 111 can be formed using any suitable technique, e.g., welding, laser welding, bonding, laser bonding, laser-assisted diffusion bonding, brazing, etc.

The device housing 102 can include or be formed from any suitable material, e.g., titanium, titanium alloys, stainless steel, noble metal coated metallic enclosures (e.g., gold), etc. The device housing 102 can be electrically conductive, electrically insulative, or both. For example, the first portion 102-1, or portions thereof can be electrically conductive while the second portion 102-2 or portions thereof can be electrically insulative.

In addition to the device housing 102 and the power source 10, the device 100 can include one or more additional components. For example, the device 100 can include mechanical structures or components such as, e.g., one or more fixation elements 108 or a flange 110. Such mechanical structures can facilitate movement and placement of the device 100 in a target environment. The fixation elements 108, which can include a plurality of fixation tines, as depicted, and/or one or more helical fixation elements, can be used or configured to retain a position of the device 100 in the target environment. The flange 110 can be used or configured to facilitate movement or manipulation of the device 100 in the target environment. For example, the flange 110 can be attached to a guidewire or other tool to position the device 100, and the fixation elements 108 can retain a position of the device once properly implanted in a patient.

The device 100 can also include various electrical components such as, for example, an electrode 112, an electromechanical device 114, control circuitry 116, or other suitable electrical components. The electrode 112 can facilitate sensing or electrical pulse delivery in a target environment, e.g., sensing cardiac electrical signals and/or delivering cardiac pacing pulses to myocardial tissue against which the electrode 112 is positioned. The electromechanical device 114 can include, for example, at least one of a kinetic energy harvester (such as one or more beams thereof), an accelerometer, a gyroscope, motors, generators, or other electromechanical devices.

The control circuitry 116 can include any suitable hardware or devices to control various electrical or electromechanical devices that the device 100 can include. In general, the control circuitry 116 can be disposed in the device housing 102 and can be operatively coupled to the power source 10. In one or more embodiments, the control circuitry 116 can include a power management system. The power management system can be disposed in the housing 102 and can be operatively coupled to the power source 10. The power management system can be configured to control charging and discharging of the power source 10. For example, the control circuitry 116 can be operatively coupled to a kinetic energy harvester (e.g., electromechanical device 114). The kinetic energy harvester can be configured to move along the device axis 2 including at least partially in or through the passageway 20 of the power source 10 and produce electrical energy as the device 100 is moved. The control circuitry 116 can be configured to receive the electrical energy provided by the kinetic energy harvester and use such electrical energy to charge the power source 12 or deliver such electrical energy to one or more electronic components of the device 100.

In one or more embodiments, the control circuitry 116 can include a controller that includes one or more processors operatively coupled to the power source 10 and disposed in the device housing 102. The controller can be configured to receive power from the power source 10 and deliver one or more therapeutic pulses. To deliver such therapeutic pulses, the controller can be operatively coupled to the electrode 112 and configured to deliver the one or more therapeutic pulses using the electrode.

The specific configuration of the control circuitry 116 can depend on a type of the device 100. The control circuitry 116 can include, e.g., one or more processors, logic gates, clocks, queues, Electro-Static Discharge (ESD) protection circuitry for input and output signals, line drivers and line decoders for interfacing to external devices, etc. The control circuitry 116 can be provided in a Field-Programmable Gate Array (FPGA), a circuit board, a system on a chip, a fixed or mobile computer system (e.g., a personal computer or minicomputer), implemented in software, etc.

The exact configuration of the control circuitry 116 is not limiting and essentially any device capable of providing suitable computing capabilities and signal processing capabilities (e.g., sensor data, control signals, battery management, etc.) can be used. Further, in one or more embodiments, data (e.g., sensor data, therapy data, etc.) can be analyzed by a user, used by another machine that provides output based thereon, etc. As described herein, a digital file can be any medium (e.g., volatile or non-volatile memory, a CD-ROM, a punch card, magnetic recordable tape, etc.) containing digital bits (e.g., encoded in binary, trinary, etc.) that can be readable and/or writeable by control circuitry 116 described herein. Also, as described herein, a file in user-readable format can be any representation of data (e.g., ASCII text, binary numbers, hexadecimal numbers, decimal numbers, audio, graphical) presentable on any medium (e.g., paper, a display, sound waves, etc.) readable and/or understandable by a user.

Devices (e.g., device 100) that include a battery (e.g., power source 10) with a passageway (e.g., passageway 20) as described herein, can provide additional space or volume for various components contained within the interior space or volume of such devices. Furthermore, the passageway of the battery can allow components to move through battery. Accordingly, components of such devices cannot be restricted to a single side of the battery within the devices.

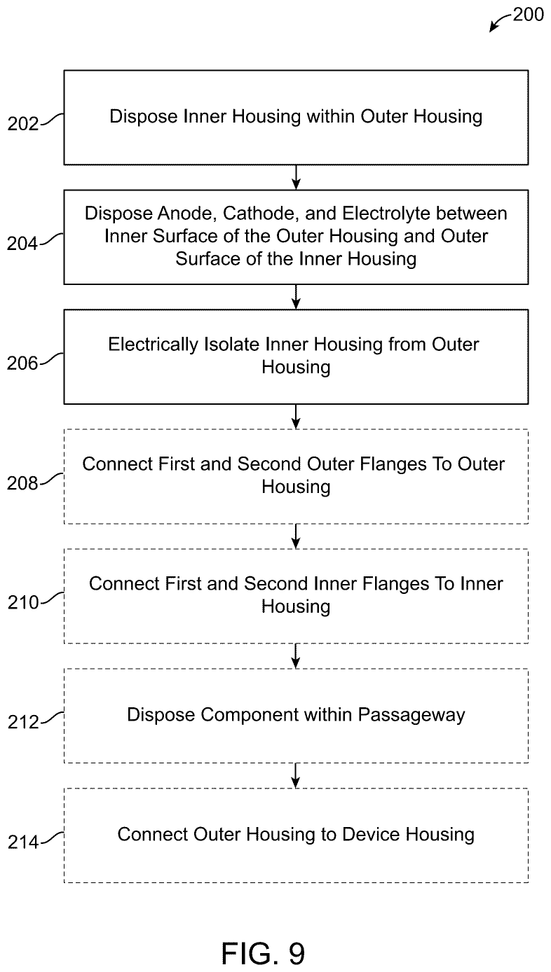

Any suitable technique can be utilized to manufacture the power source 10. For example, FIG. 9 is a flowchart of one embodiment of a method 200 of manufacturing the power source 10. Although described regarding power source 10 of FIGS. 1-5, the method 200 can be utilized to manufacture any suitable power source. At 202, the inner housing 18 is disposed at least partially within the outer housing 16. At 204, the anode 58, cathode 60, and electrolyte 62 is disposed between the inner surface 22 of the outer housing 16 and the outer surface 28 of the inner housing 18 (e.g., within cavity 56) using any suitable technique. In one or more embodiments, the anode 58 and cathode 60 can be wound around the outer surface 28 of the inner housing 18 as is further described herein, and the anode, cathode, and inner housing can be disposed at least partially within the outer housing 16 using any suitable technique. Further, the electrolyte 62 can be disposed at least partially within the outer housing 16 after either the first or second ends 12, 14 of the power source 10 have been hermetically sealed with outer and inner flanges 32, 40, 48, 50 and an insulator 52, 54. In one or more embodiments, both ends 12, 14 of the power source 10 can be hermetically sealed and the electrolyte disposed within the outer housing 16 through a fill port formed in at least one of the outer flanges, 32, 40, inner flanges 48, 50, first insulator 52, or second insulator 54.

At 206, the inner housing 18 is electrically isolated from the outer housing 16 using any suitable technique. In one or more embodiments, the first insulator 52 can be disposed between the outer and inner housings 16, 18 at the first end 12 of the device, and/or the second insulator 54 can be disposed between the outer and inner housings at the second end of the device. In one or more embodiments, the first and second outer flanges 32, 40 can be connected to the outer housing 16 at the first end 12 and second end 14 of the power source 10 respectively at 208 using any suitable technique. In one or more embodiments, one or both of the first and second outer flanges 32, 40 can be connected to the outer housing 16 either prior to or after the inner housing 18 is disposed at least partially within the outer housing. At 210 the first and second inner flanges 48, 50 can be connected to the inner housing 18 at the first end 12 and second end 14 of the power source 10 at 210 using any suitable technique. In one or more embodiments, the inner flanges 48, 50 can be connected to the inner housing 18 prior to the outer flanges 32, 40 being connected to the outer housing 16. In one or more embodiments, the first outer flange 32 can be connected to the outer housing 16 follow by the first inner flange 48 being connected to the inner housing 16 or vice versa. Similarly, the second outer flange 40 and second inner flange 50 can be connected in any order.

At 212, an element or component 30 can be disposed at least partially within the passageway 20 using any suitable technique. Further, the outer housing 16 can be connected to the device housing at 214 using any suitable technique. In one or more embodiments, the outer housing 16 can be connected to the device housing 102 such that the outer housing is hermetically sealed to the device housing 102.

In one or more examples, the described techniques can be implemented in hardware, software, firmware, or any combination thereof. If implemented in software, the functions can be stored as one or more instructions or code on a computer-readable medium and executed by a hardware-based processing unit. Computer-readable media can include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions can be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein can refer to any of the foregoing structure or any other physical structure suitable for implementation of the described techniques. Also, the techniques could be fully implemented in one or more circuits or logic elements.

What is claimed is:

1. An electrical power source extending along a longitudinal axis between a first end and a second end of the power source, wherein the power source comprises:
an outer housing extending along the longitudinal axis;
an inner housing disposed at least partially within the outer housing and electrically isolated from the outer housing, the inner housing extending along the longitudinal axis; and
a passageway defined by an inner surface of the inner housing, the passageway extending between the first end and the second end of the power source.

2. The power source of claim 1, further comprising a first outer flange connected to the outer housing at the first end of the power source and a second outer flange connected to the outer housing at the second end of the power source.

3. The power source of claim 2, further comprising a first inner flange connected to the inner housing at the first end of the power source and a second inner flange connected to the inner housing at the second end of the power source.

4. The power source of claim 3, further comprising a first insulator disposed between the first outer flange and the first inner flange, and a second insulator disposed between the second outer flange and the second inner flange.

5. The power source of claim 4, wherein at least one of the first insulator or second insulator comprises a glass feedthrough material.

6. The power source of claim 4, wherein at least one of the first insulator or second insulator comprises a diffusion bonded sapphire material.

7. The power source of claim 4, wherein at least one of the first insulator or second insulator comprises a polymer material.

8. The power source of claim 1, further comprising a kinetic energy harvester disposed at least partially within the passageway.

9. The power source of claim 1, further comprising a power management system.

10. The power source of claim 1, further comprising at least one conductor disposed at least partially within the passageway.

11. The power source of claim 1, further comprising an anode, a cathode, and an electrolyte, wherein the anode, cathode, and electrolyte are disposed between the outer housing and the inner housing.

12. An implantable medical device comprising:
a device housing extending along a device axis between a first end and a second end of the device; and
an electrical power source extending along a longitudinal axis between a first end and a second end of the power source, the power source comprising:
an outer housing extending along the longitudinal axis, wherein the outer housing defines at least a portion of the device housing;
an inner housing disposed at least partially within the outer housing and electrically isolated from the outer housing, the inner housing extending along the longitudinal axis; and
a passageway defined by an inner surface of the inner housing, the passageway extending between the first end and the second end of the power source.

13. The device of claim 12, wherein the passageway of the power source extends along the longitudinal axis.

14. The device of claim 12, wherein the passageway of the power source is substantially parallel to the longitudinal axis.

15. The device of claim 12, further comprising a kinetic energy harvester disposed at least partially within the passageway.

16. The device of claim 12, wherein the device further comprises a first outer flange connected to the outer housing at the first end of the power source and a second outer flange connected to the outer housing at the second end of the power source.

17. The device of claim 16, wherein the power source further comprises a first inner flange connected to the inner housing at the first end of the power source and a second inner flange connected to the inner housing at the second end of the power source.

18. The device of claim 17, wherein the power source further comprises a first insulator disposed between the first outer flange and the first inner flange, and a second insulator disposed between the second outer flange and the second inner flange.

19. The device of claim 18, wherein at least one of the first insulator or second insulator comprises a glass feedthrough material.

20. The device of claim 18, wherein at least one of the first insulator or second insulator comprises a diffusion bonded sapphire material.
